(19) United States
(12) Patent Application Publication
(10) Pub. No.: US 2023/0173216 A1
(43) Pub. Date: Jun. 8, 2023

(54) METHOD AND DEVICE FOR TREATING MIGRAINES

(71) Applicant: COOLTECH, LLC, Baltimore, MD (US)

(72) Inventors: Harikrishna Tandri, Ellicot City, MD (US); William DeMore, Baltimore, MD (US); Krystian Zimowski, Washington, DC (US); Brian Lipford, Belair, MD (US); Steven Schaefer, Lutherville, MD (US); Benjamin R. Lane, Hydes, MD (US); Edward R. Prescott, Baltimore, MD (US); Jonathan H. Green, Baltimore, MD (US); Kun Li, Boyds, MD (US); Thomas Daniel James, Philadelphia, PA (US); Kayleigh E. Boyle, Baltimore, MD (US); Colby Johnson, Baltimore, MD (US); Michael Damiani, Baltimore, MD (US)

(21) Appl. No.: 17/959,025

(22) Filed: Oct. 3, 2022

Related U.S. Application Data
(60) Provisional application No. 63/251,287, filed on Oct. 1, 2021.

(51) Int. Cl. A61M 16/16; A61M 16/10

(52) U.S. Cl. CPC A61M 16/16; A61M 16/1075

(57) ABSTRACT

A portable device for treatment of migraine having a desktop unit defining an airpath through a desiccant, a heat sink, an air mover and an air outlet, and a tubeset including a tube having one end for connecting to the desktop air outlet and a second end connected to a handheld unit, the handheld unit including a water compartment, and a nebulizer/transducer for introducing water droplets into the air stream. A nasal pillow is provided at the end of the handheld unit for placing against a user's nose.

### BACKGROUND OF THE INVENTION

The invention relates generally to methods and devices for treating migraines.

### SUMMARY OF THE INVENTION

The present inventions include methods and handheld devices for treating migraines, headaches, as well as other pain and mood disorders. The inventors have unexpectedly discovered that a modest supplemental stream of air (supplemental to ordinary respiration) directed to the region of the nasopharynx where the sphenopalatine ganglion resides results in an unexpected reduction in the symptoms of migraine. According to a preferred embodiment, the supplemental flow of air may be 2 Liters per min (“L/min”) to 24 L/min, preferably 4 L/min to 12 L/min and most preferably 6 L/min, all in addition to the user's natural respiration.

Accordingly, there is presented according to the invention a method for the treatment of migraine, headaches, as well as other pain and mood disorders comprising delivering a supplemental stream of air to the back of the nasopharynx which causes a calming in the activity of the Sphenopalatine ganglion, resulting in a reduction in the symptoms of migraine, headache, pain or other mood disorder.

According to various embodiments of the invention, the incrementally higher flow of air (compared to normal respiration) may be accompanied by an independent supply of nebulized water droplets to the nasal turbinates.

There is also presented according to the invention a handheld device which may be used by a migraine sufferer to self-treat the symptoms of a migraine. The handheld personal device takes ambient/environmental air through an intake port; optionally dehumidifies the air, and forces the air through a tube and out the end of the tube where the patient directs the flow of air into one nostril.

According to a preferred embodiment of the invention, the device would have a single nostril port and would blow the air into one nostril with the air exiting the other nostril, but could also exit partially or totally from the mouth.

According to one embodiment, the tube may have a tapered end adapted to be placed against or inserted a short way into and make a gentle seal with the patient's nostrils. According to another embodiment, the distal end of the tube may be adapted to receive a replaceable/removable/disposable nostril interface which is used to form the interface and seal with the patient's nostril. The air is preferably pulled into the device and out the exit port via an internal fan or centrifugal pump and may be configured to operate via connection to standard wall socket and/or optionally by onboard batteries.

According to a preferred embodiment of the device, it may be configured to deliver a fine mist of a physiologically balanced solution, as well as tap water, to the subject's nasopharynx, simultaneously or intermittently with the administration of the air flow, which improves the tolerability of the air flow.

According to a preferred embodiment of the invention, the device may have a handheld unit attached to the distal end of the tube, the handheld unit adapted to be comfortably held by the patient to his or her face, with the nostril interface placed at his or her nostril. The handheld unit may contain a nebulizer or ultrasonic transducer for nebulizing a source of a physiologically balanced solution or water for delivery via the nostril interface. The handheld unit may include a water/liquid reservoir for holding a supply of physiologically balanced water, saline and/or drug solution. The reservoir may be in the form of a replaceable cartridge, in which case the handheld unit is configured with cartridge port for receiving a replaceable cartridge.

According to another embodiment, the device may be configured to deliver medications to the patient's nostril. The types of medications that may be delivered using a device of the invention would include any medications that are delivered to a patient through the nose, for example via a nasal mist or spray. Lidocaine is an example of a drug that is sold in nasal sprays to treat migraines.

According to another embodiment of the invention, the handheld unit may have a second, medicine reservoir or cartridge port for receiving a replaceable medicine cartridge configured to contain a medication. For example, the device might be used to alternately or simultaneously deliver air and a spray of water, saline and/or medication, such as lidocaine, to the patient's nostril. The combined effect of the air flow and the numbing effect of the lidocaine provide an unexpected boost in relief from migraine pain.

According to a further embodiment of the invention, the flow air from the device may be used as a vehicle to deliver or push a lidocaine solution or other numbing or topical anesthetic drug all the way to the nasopharynx where the SPG nerve bundle is located to deaden the response from the migraine. The location of the SPG nerve bundle is at the very back of the airway, isolated and difficult to physically access. Accordingly, a device according to the invention may be used to deliver a supplemental flow of air that can transport or push the lidocaine back into the very back of the nasal cavities and/or to the nasopharynx and/or very back of the throat. According to a preferred embodiment, the flow of air necessary to push lidocaine or other drug to the back of the airway is 2 Liters per min (“L/min”) to 24 L/min, preferably 4 L/min to 12 L/min and most preferably 6 L/min, all in addition to the user's natural respiration.

The air stream and the physiologically balanced water, saline or other suitable liquid are provided to the patient via isolated delivery paths using a specially designed device, tubes, and mask which deliver the air and the water to the patient at or just before a delivery point at the nostril opening, up to which point the separate air and water flow paths have been kept isolated from one-another. No portion of the device is required to be inserted into the nasal cavity.

According to preferred embodiments of the invention, the ambient air need not be heated or cooled, dried or humidified prior to delivery to the subject.

According to various embodiments, the water may be misted using an atomizer or nebulizer, typically via an ultrasonic transducer. In any event, the resulting water particles are one or more orders of magnitude larger than the water particles in humidified air or water vapor; accordingly, the great majority of the administered water does not undergo a phase change during or immediately after administration to the patient, and the mist has little to no material effect on the water content of the supplemental flow air, which is to say that the administration of liquid water does not humidify the air by more than 0% to 5% humidity in excess of the humidity of the air before it passes the misting point.

The device draws ambient air through a filtered inlet plenum and optionally dries the air for delivery to the patient/user. Once inside the device, the air path passes through an inlet plenum pathway, a bulk plenum pathway, a desiccant cartridge, a heat sink, across various sensors and out an air outlet. While inside the device, the air flow path is isolated from entrance to exit. The device also has a separate water delivery system which keeps the water flow path isolated from the air flow path. The handheld unit has a dual function of storing and nebulizing the water/liquid supply, and delivering the nebulized liquid into the air flow just before delivery to the patient.

According to a preferred embodiment, the device has a portable housing having an air inlet and air outlet connected by an air flow path through the device, a fan situated in the housing to draw air from an air supply through the air inlet, through the air flow path and out the air outlet into an air delivery tube, a fluid pump or nebulizer/atomizer situated in the handheld unit to draw fluid from the water/liquid reservoir/cartridge and delivering it to the nostril interface. The device also includes temperature, humidity, pressure, and flow sensors for the air flow, and one or more batteries along with standard connections for wall power (110V-240V). In addition, the device interface may be configured to allow an operator to manually select the air flow rate (from low to high, in multiple increments), as well as the volume of water/saline/liquid delivery (from low to high, in multiple increments).

According to a preferred embodiment of the invention, the device is small and light, approximately the size of a TV remote, easily placed in a pocket or purse.

### BRIEF DESCRIPTION OF THE DRAWINGS

The foregoing summary, as well as the following detailed description of the preferred invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a perspective view of a migraine treatment device according to an embodiment of the invention.

FIG. 2 shows top cover and bottom components of the embodiment of FIG. 1.

FIG. 3 shows the desiccant cartridge and its connection to the bottom component of the embodiment of FIG. 1.

FIG. 4 is a cutaway view of the embodiment of FIG. 1 showing the heat sink portion.

FIG. 5 shows the vacuum manifold and flow restriction area of the embodiment of FIG. 1.

FIG. 6 shows the blower and blower interface of the embodiment of FIG. 1.

FIG. 7 is a cutaway view of the bottom inside portion of the embodiment of FIG. 1.

FIG. 8 shows cutaway views of a tubeset and handheld unit according to an embodiment of the invention.

FIG. 9 shows the primary device PCB mounted on the bottom side of the device cover of the embodiment of FIG. 1.

FIG. 10 shows the button layout of the embodiment of FIG. 1.

FIG. 11 shows the location of the misting PCB according to the embodiment of FIG. 1.

FIG. 12 shows the location of the EEPROM PCB according to the embodiment of FIG. 1.

FIG. 13 shows the location of the handpiece mounting to the main device according to the embodiment of FIG. 1.

Features in the attached drawings are numbered with the following reference numerals:

| | |
|---|---|
| 1 Desiccant Cartridge | 2.11 Bottom Blower Damping Mount |
| 1.1 Desiccant Cartridge Top | 2.12 Top Blower Damping Mount |
| 1.2 Desiccant Cartridge Bottom | 2.13 Blower |
| 1.3 Entry Filter Grate | 2.14 Pressure Manifold |
| 1.4 Desiccant Inlet Filter | 2.15 Thermistor |
| 1.5 Desiccant beads | 2.16 Temperature/Humidity Sensor |
| 1.6 Bottom Filter Grate | 2.17 flexible tube |
| 1.7 Desiccant Outlet Filter) | 2.18 Base Enclosure Doghouse |
| 1.8 Desiccant Cartridge Foam Gasket | 2.19 Tubing |
| 2 Durable Unit (main housing) | 2.20 Absolute pressure sensor |
| 2.1 Desiccant Connection Cap | 2.21 Device Outlet Filter |
| 2.2 Desiccant Connection Cup | 2.22 Base Enclosure Capture Plate |
| 2.3 Thread Forming Screw for Plastic | 2.23 Desiccant Connection O-Ring |
| 2.4 Top Vacuum Manifold | 2.24 Heat Sink O-Ring |
| 2.5 Vacuum Manifold, Bottom | 2.25 RTV Silicone Sealant |
| 2.6 O-ring | 3 Tubeset |
| 2.7 Heatsink | 3.1 Air Tube |
| 2.8 Flow Restrictor | 3.2 Flow Conduit |
| 2.9 Differential Pressure Sensor Tube | 3.3 Ultrasonic Transducer |
| 2.10 Differential pressure sensor | 3.4 Transducer Gasket |

### DETAILED DESCRIPTION OF THE INVENTION

Unless defined otherwise, all terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, the term “ambient air” means the breathable air present in the environment where the device is being used, including the ambient temperature (without active heating or cooling) and ambient humidity (without active humidification or dehumidification), and, except for removal of particulate pollutants, without any addition of any other gas or vapor or removal of any of the gaseous components of pure air (other than minute amounts of gases and other materials that might be removed by the desiccant).

The invention utilizes a supplemental/incrementally higher flow of air over the user's natural rate of respiration, which includes single-nostril flow rates of between 2 Liters per min (“L/min”) to about 24 L/min, preferably from about 4 L/min to about 12 L/min and most preferably about 6 L/min, all over to the user's natural rate of respiration, whether at rest or during or after exertion.

According to a preferred embodiment of the invention, the administration of the supplemental flow of ambient air to the SPG within the pterygopalatine fossa, accompanied by the simultaneous delivery of an isolated supply of misted or nebulized water, surprisingly results in significant amelioration of pain and other symptoms from migraine.

In preferred embodiments of the invention, the water is a saline solution that approximately matches the saline content of the human body, supplied at temperatures from ambient temperature, to normal body temperature (e.g., for humans, 37° C.), and even as warm as a highest clinically acceptable temperature, with water at the ambient environmental temperature being particularly preferred. The invention may use water temperatures from (ambient temperature) C up to about 40° C., as 40° C. is considered a clinically acceptable temperature.

According to various embodiments, the invention monitors the flow and/or amount of ambient air supplied to the patient's nostrils and simultaneously supplies, via an isolated delivery path and misting nozzles located at the opening of the patient's nostrils, the amount of misted water administered.

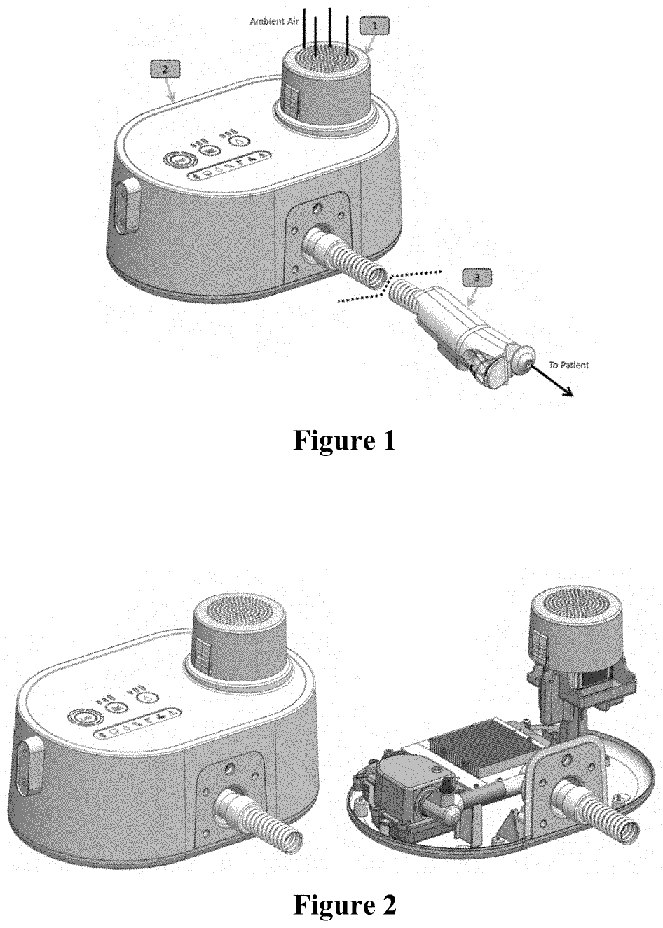

Referring to FIGS. 1 and 2, the device according to the invention includes a portable desktop unit having an air mover, such as a fan, a removable desiccant cartridge, air temperature, flow rate, humidity and pressure sensors, input console including on/off and flow rate controls, a display, a controller, and an air pathway between an ambient air inlet and an air tube interface/port which passes through the fan and the desiccant cartridge.

The device further includes a handheld unit connected to the desktop unit by an air tube. The handheld unit includes a water/saline reservoir and an air pathway between the tube interface/port and the nasal interface, which is preferably a disposable silicone nasal pillow situated on a short air outlet stub, preferably at or near the top of the handheld unit. A water delivery path connects the water/saline reservoir with the air outlet stub, and gravity or a small pump forces fine stream of water from the reservoir toward the outlet stub. An ultrasonic transducer is connected to the water rendering it into a mist of water droplets, optimally sized around 3 um to 8 um in diameter, just as the water enters the air outlet stub. According to an alternative embodiment, the fine stream of water passes through a nebulizer just before it enters the air outlet stub.

The device may have a standard power cord, battery compartment or both.

To use, the patient presses the power button on the input console of the desktop unit, optionally selects a preferred air and saline flow rate. The fan powers on, draws air through the ambient air inlet, and forces it through the desiccant at a rate sufficient for the air to leave the outlet of the desktop unit and enter the air tube at the selected flow rates. After a few second delay, the nebulizer/atomizer/transducer in the handheld unit powers on and delivers atomized/nebulized water droplets into the air stream as it passes the location of the nebulizer/atomizer/transducer. The user places the nasal pillow at one of his or her nostrils, and the supplemental volume air flows to the back of the patient's nasopharynx accompanied by the water droplets to effectively treat the area adjacent the SPG nerve bundle. The device may use the pressure and temperature air sensors to ensure the delivered flow to the patient is safe from excessively high pressure or temperature.

The basic structure, function and methods of use of the invention having been described above, a non-limiting preferred embodiment named the MiHelper shall now be described in detail.

Device Overview

The MiHelper is a device used for alleviating the pain and symptoms associated with migraines, designed for use at home. The device draws air from the ambient environment into the process air pathway, which is then dried, cooled, and delivered to the patient with the introduction of saline mist. This process, known as transnasal cooling, is used to extract energy from the nerve bundles associated with migraine pain in the upper airway of the patient. The user interface consists of three buttons to start the device and to manage flow and saline deposition rates.

Design Descriptions

There are three main components to the MiHelper: Desiccant Cartridge 1, Device 2, and Tubeset 3, with the general flow path described below with reference to the figures. The device description herein is broken up into airpath and non-air path assemblies, outlining key design features along the way. The term “airpath” as used herein refers to the air being delivered to the patient and the path it takes through the device.

Airpath Components

FIGS. 1 and 2 illustrate all the components in the airpath of the MiHelper. The airpath begins at the intake of the desiccant cartridge 1.

Desiccant Cartridge and Device Connection Interface

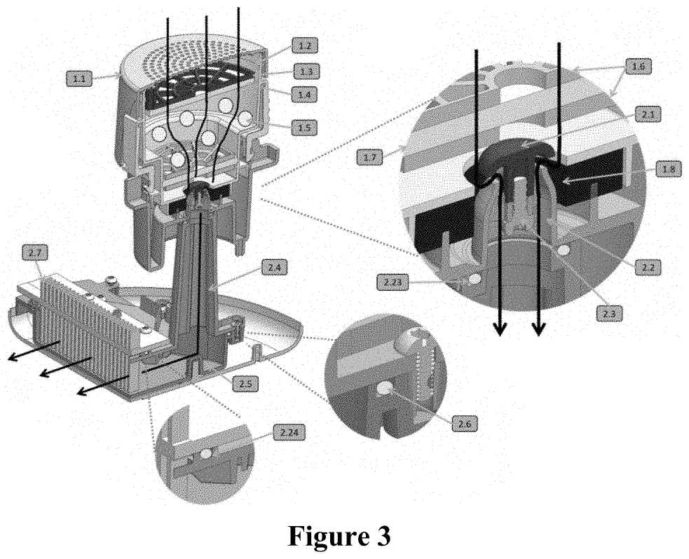

The desiccant cartridge 1 consists of an ultrasonically welded assembly filled with an intake filter 1.4 to screen out larger particulates, desiccant beads 1.5 to dry out passing air, and a hydrophobic, bacterial, and viral outlet filter 1.7. See, e.g., FIG. 3. The cartridge 1 may be inserted into the device 2 by pressing down on the spring-loaded ejection plate until the tabs on the cartridge engage and lock to their respective slots in the device. Once the cartridge is inserted, the airpath is sealed due to the compression of a gasket 1.8 by the ribs in the device, forcing ambient air through the cartridge and its elements. Notwithstanding the above-described embodiment, however, any kind of replaceable desiccant and desiccant compartment may be used, including but not limited to a tea bag-type desiccant placed into a tea bag desiccant compartment in the device.

The desiccant cartridge may optionally contain an EEPROM PCB that connects to a respective PCB on the device to ensure the user is protected against the use of fraudulent, used, or expired desiccant cartridges.

When the cartridge is not present, the device maintains its ingress protection from the airpath through a connection cap 2.1. If water were to drip in the desiccant cup 2.2 ejection plate area, there is a port connected to a tube to drain any collected water out through the bottom of the device.

Vacuum Manifold

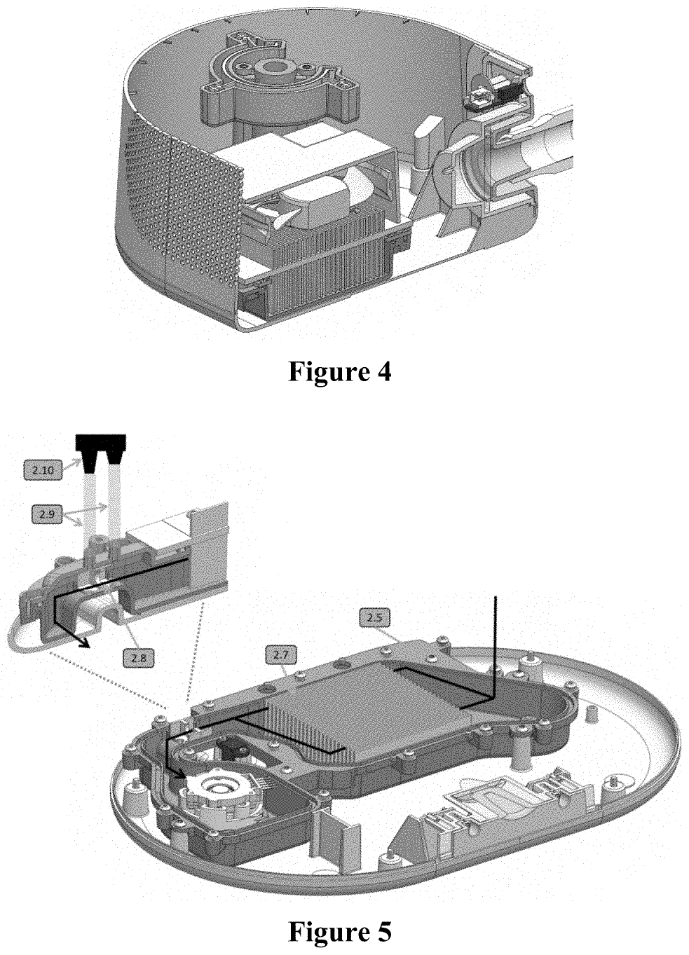

The vacuum manifold is constructed using two plastic pieces 2.4, 2.5 and a heat sink 2.7. See, e.g., FIG. 4. All the interfacing components, including the one to the cartridge cup 2.2, are connected using O-rings 2.6 to maintain a seal in the airpath. When the ambient air is forced through the desiccant beads 1.5, the air may be heated to temperatures as high as 50° C. or more, and thus needs to be cooled back down to ambient temperature. This is achieved through the aluminum heatsink 2.7.

The heatsink 2.7 is mounted on top on the vacuum manifold assembly and seals the airpath through a hollow O-ring 2.6. Heat captured by the ribs in the airpath is conducted to the ribs on the outside of airpath, which is in turn cooled by a fan mounted on top. The fan has its own air intake separate from the desiccant cartridge 1. This intake does not have any filters since it will not be exposed to the patient airpath, but it does provide ingress protection: the intake holes are angled upwards to prevent liquid ingress and are small enough for protection against solids for at-home use.

After the air is cooled by the heat sink 2.7, it passes through a flow restrictor 2.8 with barbs on either side to connect to a differential pressure sensor 2.10. See, e.g., FIG. 5. This differential pressure measured by this sensor is empirically calculated by the device software to the flow rate.

Blower+Pressure Manifold

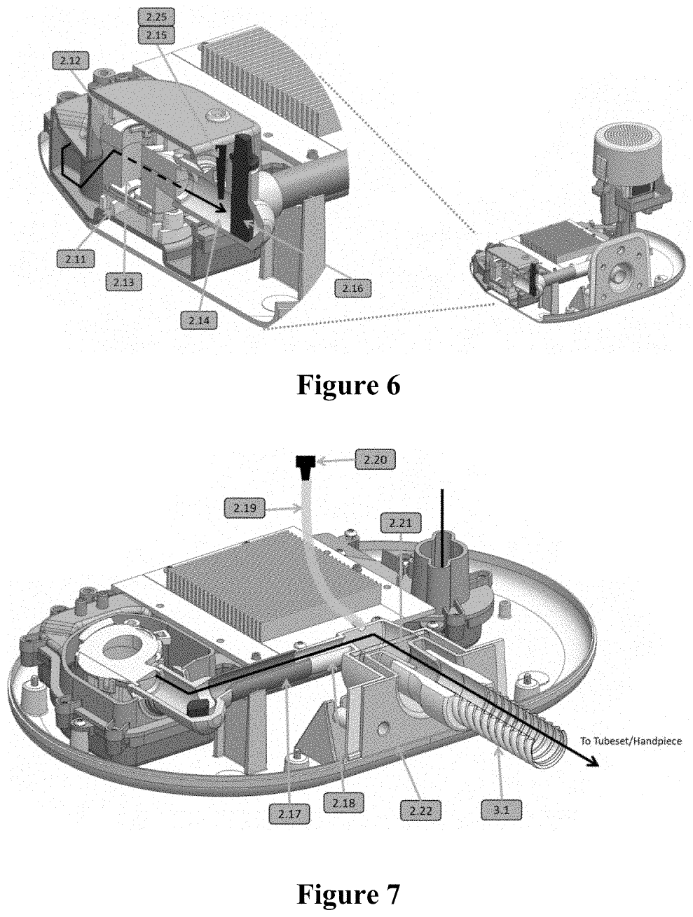

The blower 2.13 is mounted at the end of the vacuum manifold assembly, sandwiched between two silicone mounts 2.11, 2.12 for vibration isolation. See, e.g., FIG. 6.

The blower 2.13 is directly connected to the pressure manifold 2.14 which has two sensors in the airpath potted with RTV silicone sealant: a thermistor 2.15 and a humidity sensor 2.16. Both are used to calculate the patient enthalpy limits and to verify the desiccation of air by the cartridge.

Doghouse Assembly

The positive pressure side of the airpath components consists of the pressure manifold 2.14, as mentioned in the previous section, and the doghouse assembly 2.18. See, e.g., FIG. 7. They are connected by a piece of silicone tubing 2.17 and sealed by RTV silicone 2.25 sealant at the joints. At the doghouse assembly 2.18, there is a port which connects to the pressure sensor 2.20 on the board via tube 2.19. This purpose of this sensor is to detect over-pressurization and cutoff delivery of air flow at around 30 cm H₂O for patient safety. Before exiting the device into the tubeset 3, the air passes through a redundant bacterial and viral filter 2.21, serving the same purpose as the outlet filter on the desiccant 1. This filter 2.21 is also hydrophobic, so it provides water ingress protection from any saline that could drip down the tubeset into the device as well as external liquid when the tubeset is not connected.

Tubeset and Handpiece

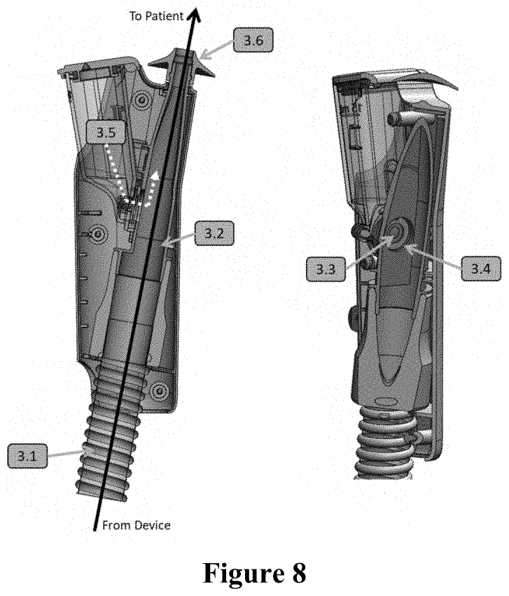

The incoming air from the device 2 passes through the tube 3.1 and handpiece (FIG. 8) before finally entering the patient's nasal passage. The nasal pillow 3.6 is the only piece in contact with the patient upon therapy. Saline gets injected in the form of mist through the ultrasonic transducer 3.3 into the airstream at the flow conduit 3.2, as shown by the white dotted line in FIG. 8. The transducer 3.3 is powered by an electrical cable running along the outer part of the tube that connects to the main device. The saline is stored in the adjacent saline reservoir 3.5 which the user can fill to the prescribed amount and close it off from the outer environment with a lid-mounted gasket. All the items described in this handpiece, including the tube, are captured in a cosmetic outer shell.

Non-Airpath Components

PCBs

There are three custom Printed Circuit Boards in the device: the main PCB, the misting PCB on the front of the device, and the EEPROM PCB for the desiccant cartridge. There is also one off-the-shelf PCB that is required to run the blower, and a corresponding EEPROM PCB on the desiccant cartridge.

Main PCB

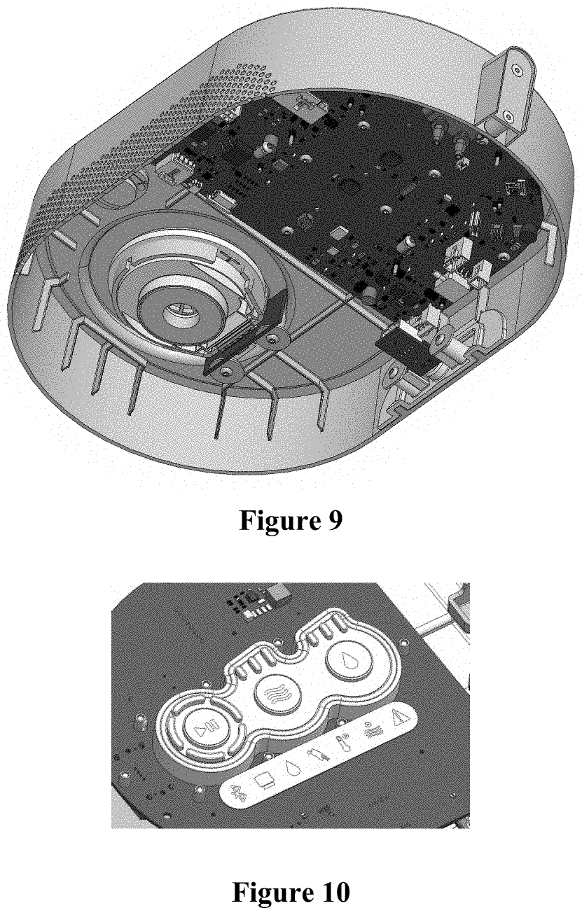

The main PCB is mounted on the underside of the device (FIG. 9) to interface with the operating buttons on the top side of the device, of which there are three: (1) Play/Pause, (2) Air flow adjustment and (3) Saline spray adjustment (FIG. 10). The buttons act like a gasket when the PCB is mounted to the outer shroud of the device and provide ingress protection, and the large number of mounting locations on the PCB provide adequate support for user button interaction.

Misting PCB

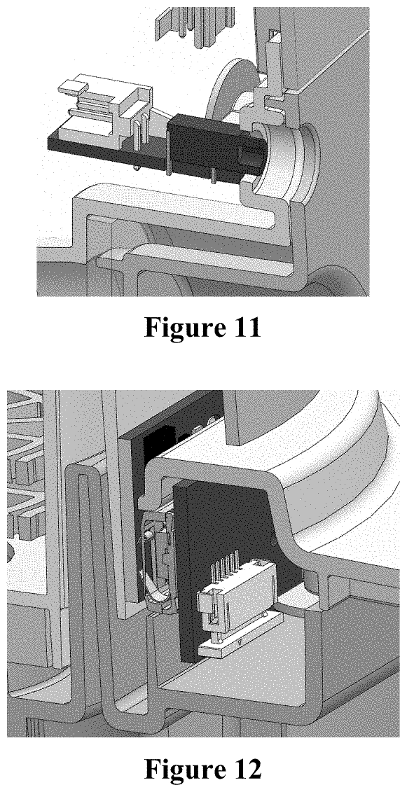

The misting PCB is mounted using two screws and washers in the doghouse assembly and provides the electrical connection to the transducer in the handpiece through the form of an auxiliary port. See, e.g., FIG. 11. However, the port is only 2.5 mm, undersized compared to the typical 3.5 mm headphone cable, to minimize any user misuse and possible damage to the device.

EEPROM PCB

The EEPROM PCB is mounted perpendicular to the plastic with screws and washers, similar to the misting PCB. See, e.g., FIG. 12. When the desiccant cartridge is inserted, the leaf springs on the device PCB compress to form a connection to the corresponding cartridge PCB.

Handpiece Mount

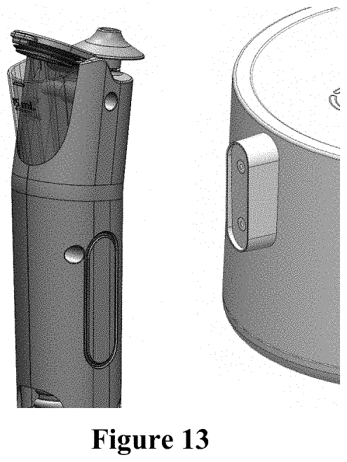

The handpiece mount (FIG. 13) stores the handpiece when not in use by the patient thanks to magnets on both the device and handpiece side. There are corresponding symbols to aid the user in mounting correctly. The orientation of the mounting also allows the user to keep the handpiece upright, operate the reservoir lid, and fill the saline reservoir to the prescribed amount without spilling.

Air Pathway Description

Air enters the process air pathway through the Desiccant Cartridge Top 1.1. The desiccant cartridge is inserted into the durable unit prior to air flow activation and a seal is created between the Desiccant Cartridge Foam Gasket 1.8 and the Desiccant Connection Cup 2.2. Ambient air is drawn through an array of filters and desiccant beads 1.5 before passing through the Desiccant Connection Cup and Vacuum Manifold of the durable unit. A seal is made between the top and bottom Vacuum Manifold parts through an O-ring 2.6. Error! Reference source not found. FIG. 3-2 shows the air passing through a heatsink in the manifold. See FIG. 3-3 for a continuation of the flow path.

FIG. 3-3 shows the top-down view (with the Top Vacuum Manifold removed, except where the tubing barbs are shown in the top left) of the air pathway through the Heatsink 2.7 and the Flow Restrictor 2.8. There are two barbs in the Top Vacuum Manifold 2.4 on either side of the Flow Restrictor that connect to a differential pressure sensor 2.10 through tubing. The air then passes to the blower 2.13, which is depicted in FIG. 3-4.

The blower, held in place by two silicone rubber mounts (Bottom Blower Damping Mount 2.11 and Top Blower Damping Mount Item 2.12), forces the process air through the Pressure Manifold 2.14, which has two mounted sensors: the Thermistor 2.15 and the Temperature/Humidity Sensor 2.16. The thermistor is potted into place using RTV Silicone Sealant 2.25.

The final stages of the durable unit process air pathway include a flexible tube 2.17 that attaches the Pressure Manifold to the Base Enclosure Doghouse 2.18. A barb on this Base Enclosure Doghouse allows for an absolute pressure sensor 2.20 to be connected via tubing 2.19. The air exits the device through the Device Outlet Filter 2.21, which is captured by the Base Enclosure Capture Plate 2.22 and passes to the tube-set, which is terminated with the handpiece.

The incoming air from the device passes through the Air Tube 3.1 and handpiece before finally entering the patient's nasal passage. As the air passes through the handpiece, it contacts the Flow Conduit 3.2, the Ultrasonic Transducer 3.3, the Transducer Gasket 3.4, and the Nasal Pillow 3.6. Saline in saline reservoir 3.5 gets injected as a mist into the airstream via the Ultrasonic Transducer 3.3, as shown by the white dotted line in FIG. 3-6.

The following important points apply to the air flow path described in this document:
The air delivered to the patient is filtered at 3 separate locations:
   Desiccant Inlet Filter at entrance to the desiccant cartridge 1.4
   Desiccant Outlet Filter after passing through desiccant material 1.7
   Device Outlet Filter upon exiting the device and entering the tubeset 2.21
The first two filters 1.4 and 1.7 are captured within the single-use disposable desiccant cartridge, and as such, it is changed after each use.
The Device Outlet Filter 2.21 is changed on a regular maintenance interval.
After passing through the blower, for the remainder of the air flow pathway the air is operating at higher pressure than the ambient environment (this is required for air flow). The pressure sensor acts as a safety mechanism, ensuring the pressure does not rise to a level unsafe for the patient.
The placement of the pressure sensor is conservative, as it is at the beginning of the tube-set. The air flow will drop in pressure over the course of the tube-set (due to tube-set resistance), and thus the measured value in the device will always be higher than the patient experiences. Therefore, pressure safety limits implemented by the device are conservative.
The placement of the temperature sensor is conservative, as it is at the device outlet, before the tube-set. In most environments the process air will experience a temperature drop as it passes through the tubing, as the air inside the device is always either hotter than or equal to ambient temperature. As the air passes through the tubeset it is surrounded by ambient air, which will be cooler than or equal to airstream temperature. Some amount of energy will be transferred to ambient through the tubing walls, cooling the air stream. As such, the measured temperature value is always conservative.
Two temperature sensors are provided for redundancy.
After passing through the desiccant cartridge the air flow will be both heated above ambient conditions (due to the release of latent heat by water molecules adsorbed by the desiccant) and drier than ambient conditions. As such, condensation inside the device is not possible (both elevated temperature and drier air prevent condensation).

It will be appreciated by those skilled in the art that changes could be made to the preferred embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

What is claimed is:

1. A device for ameliorating symptoms associated with migraine comprising, in an integrated unit:
an air delivery subassembly in a desktop unit comprising:
an air inlet,
an air outlet,
an air flow path connecting said air inlet and said air outlet,
a blower situated in said air flow path and configured to draw ambient air from said air inlet and force said ambient air through said air flow path and out said air outlet,
a desiccant chamber situated in said air flow path and configured to receive a removable and replaceable desiccant element;
a heat sink situated in said air flow path and configured to remove heat added to air in said air flow path during removal of moisture from said air by said desiccant element;
an air outlet sensor configured to measure air temperature, air flow rate, air pressure or a combination thereof;
a handheld unit comprising:
an air inlet port and tubing;
a handheld unit outlet at the distal end of the tubing;
an air path between said air inlet port of desktop unit and said handheld unit outlet;
a water compartment, including a closable access for the introduction of water;
a water path between said water compartment and said handheld unit outlet;
a nebulizer or ultrasonic transducer located and configured to convert water in said water path to water droplets just before the water enters the handheld unit outlet.

2. A device according to claim 1, further comprising a tube configured to attach at one end to said air outlet of said desktop unit and configured to attach at a second end to said air inlet port of said handheld unit.

3. A device according to claim 1, further comprising a disposable nasal pillow configured to fit to a distal end of said handheld unit outlet.

4. A device according to claim 1, wherein said water path is configured to deliver water from said water compartment to said handheld unit outlet under the force of gravity.

5. A device according to claim 1, further comprising a water pump configured and located to force water from said water compartment through said water path.

6. A method for the amelioration of pain due to migraine comprising:
delivering to a subject's nostril ambient air at a continuous flow rate of about 2 Liters per min (“L/min”) to about 24 L/min,
simultaneously delivering to said subject's nostril water in the form of a mist, and
maintaining isolation between said ambient air and said water until just prior to delivery of said ambient air and said mist of water to said subject's nostril.

7. A method according to claim 6, wherein said continuous flow rate is about 4 L/min to about 12 L/min.

8. A method according to claim 6, wherein said continuous flow rate is about 6 L/min.
